(19) United States
(12) Patent Application Publication
(10) Pub. No.: US 2010/0040679 A1
(43) Pub. Date: Feb. 18, 2010

(54) Compositions for Reducing Nicotine Withdrawal Symptoms and/or Tobacco Usage

(76) Inventors: Shing Yue Chang, Parsippany, NJ (US); Charlotte A. Lemmonds, Parsippany, NJ (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/444,208

(22) PCT Filed: Oct. 8, 2007
(86) PCT No.: PCT/US07/80678

§ 371 Date: Apr. 3, 2009

Related U.S. Application Data
(60) Provisional application No. 60/828,682, filed on Oct. 9, 2006.

(51) Int. Cl. A61K 31/4439; A61K 31/13; A61K 31/137; A61K 31/485; A61K 9/64; A61K 9/68; A61P 25/34

(52) U.S. Cl. 424/456; 514/661; 514/649; 514/289; 514/343; 424/48

(57) ABSTRACT

Compositions useful for treating an individual with nicotine dependence comprising a combination of an α₃β₄ nicotinic receptor antagonist and a nicotine metabolite are disclosed. More particularly, compositions comprising cotinine or a pharmaceutically acceptable salt thereof are disclosed. Methods of alleviating nicotine withdrawal symptoms and/or tobacco usage by administration of these compositions are also disclosed.

### FIELD OF THE INVENTION

The present invention relates to compositions useful for treating nicotine dependence comprising a combination of an α₃β₄ nicotinic receptor antagonist and a nicotine metabolite. More particularly, the invention relates to compositions comprising: dextromethorphan, dextrorphan or pharmaceutically acceptable salts thereof; and cotinine or pharmaceutical acceptable salts thereof. The invention also relates to methods of using such compositions for reducing nicotine withdrawal symptoms and reducing tobacco usage.

### BACKGROUND OF THE INVENTION

It is generally known that active as well as passive smoking of tobacco products, such as cigarettes, cigars and pipe tobacco presents serious health risks to the user and those subjected to secondary smoke. It is also known that the use of smokeless forms of tobacco, such as chewing tobacco, spit tobacco and snuff tobacco, presents serious health risks to the user. Furthermore, the use of tobacco products in public areas is increasingly either restricted or socially unacceptable. Consequently, smokers and other tobacco users often try to quit the habit. Others may attempt to cut back on the amount of tobacco used in order to reduce health risks.

Although the damaging effects of tobacco usage are well known, most individuals who are nicotine dependent have great difficulty in overcoming their dependence. The difficulty arises in part due to the highly addictive nature of nicotine and the strong nicotine withdrawal symptoms that can occur when one begins to deprive the body of the nicotine to which it has grown dependent. Indeed, overcoming nicotine withdrawal symptoms is a critical challenge for those attempting to conquer nicotine dependence.

Nicotine addiction is a complex process which includes pharmacological, psychological and social factors. One pharmacological mechanism of nicotine addiction is the activation of the nicotinic acetylcholine receptor causing the release of dopamine in the mesolimbic pathway of the brain. Although the release of dopamine is strongly associated to addiction, the release of other neurotransmitters (such as acetylcholine, norepineneprhine, serotonin, glutamate and others) may contribute to nicotine addiction as well.

Nicotine withdrawal symptoms, particularly nicotine cravings, may arise in several ways. For instance, studies have shown that following a quit attempt, smokers report moderate levels of steady nicotine craving throughout the day. This could lead to relapse and a return to tobacco usage for those attempting to quit. In addition to steady cravings, smokers may also experience episodic, or acute, cravings. These acute cravings may be provoked by a number of stimuli, such as exposure to smoking related cues, seeing smoking paraphernalia or others engaged in smoking, or inhaling second hand smoke. Such episodic cravings may also lead to relapse if proper coping measures are not employed.

In an attempt to assist those who wish to eliminate or reduce tobacco usage, efforts have been made to provide those in need with some level of nicotine craving relief. Historically, these efforts have predominantly focused on the activity and administration of nicotine itself. This nicotine replacement therapy (NRT) helps to combat the intense nicotine withdrawal symptoms encountered by many individuals upon quitting smoking or other tobacco usage. These therapies are traditionally offered in the form of nicotine-containing chewing gums, lozenges or transdermal patches. While such means are useful as aids to reduce or quit smoking, there is an ongoing need to provide improved or alternative forms of NRT, as well as therapies that provide alternatives to nicotine-containing products.

Dextromethorphan is generally available over the counter as a cough suppressant. Dextromethorphan and its active metabolite, dextrorphan are also known antagonists of the N-methyl D-apartate (NMDA) receptor. See WO 00/16762 to Caruso. In addition, dextromethorphan and dextrorphan have been shown to act as α₃β₄ nicotinic receptor antagonists and, thus, block the neural nicotinic receptors in the central and autonomic nervous system. See US 2002/0103109 to Glick. It has been speculated that each antagonistic activity may contribute to the usefulness of dextromethorphan and dextrorphan for reducing nicotine, as well as other substance dependence. See Caruso and Glick. However, the benefit of treating nicotine addiction with dextromethorphan may be offset by the side effects associated therewith, such as drowsiness, and the potential for abuse.

Glick et al. relates to methods and compositions for treating addiction disorders by administration of a first and a different second α₃β₄ nicotinic receptor antagonist.

Caruso relates to methods for reducing nicotine dependence by administering at least one nicotine-dependency reducing agent selected from the group consisting of dextromethorphan, dextrorphan and pharmaceutically acceptable salts thereof.

The use of nicotine metabolites for treating nicotine addiction has also been discussed. US 2005/0100902 to Grattan relates to vaccines for treating nicotine addiction comprising a metabolite of nicotine, which is used for immunotherapy of nicotine addiction. US 2006/0112965 to Whalen relates to a chewing tobacco substitute which comprises a non-tobacco leaf component, an alkaline component and a nicotine compound such as nicotine polacrilex or cotinine.

Not wanting to be bound by theory, it is believed that combining an α₃β₄ nicotinic receptor antagonist, such as dextromethorphan, with a metabolite of nicotine, such as cotinine, at low levels, provides a suitable composition for reducing nicotine withdrawal symptoms or tobacco usage while avoiding the potential side effects often associated with dextromethorphan administration.

The present invention relates to combination therapies comprising α₃β₄ nicotinic receptor antagonists and metabolites of nicotine for treating nicotine addiction. More particularly, the present invention relates to compositions comprising dextromethorphan, dextrorphan or pharmaceutically acceptable salts thereof; and cotinine or pharmaceutically acceptable salts thereof. The present invention also relates to methods of reducing nicotine withdrawal symptoms and tobacco usage by administering compositions comprising dextromethorphan, dextrorphan or pharmaceutically acceptable salts thereof, and cotinine or pharmaceutically acceptable salts thereof.

### SUMMARY OF THE INVENTION

The present invention relates to compositions suitable for reducing nicotine withdrawal symptoms or tobacco usage which comprise an α₃β₄ nicotinic receptor antagonist and a nicotine metabolite. In one embodiment the α₃β₄ nicotinic receptor antagonist is selected from dextromethorphan, dextrorphan or their pharmaceutically acceptable salts. In one embodiment the metabolite of nicotine is cotinine or pharmaceutically acceptable salts thereof. In another embodiment, the composition comprises low doses of dextromethorphan, dextrorphan or a pharmaceutically acceptable salt thereof and cotinine or pharmaceutically acceptable salts thereof. The present invention also relates to methods of reducing nicotine withdrawal symptoms and/or reducing tobacco usage by administration of the compositions described herein.

### BRIEF DESCRIPTION OF THE DRAWINGS

FIG. 1 depicts overall mecamylamine-precipitated nicotine abstinence signs over 30 minutes in subjects pretreated subcutaneously with 1 mg/kg dextromethorphan, 1 mg/kg cotinine, a combination of dextromethorphan and cotinine each as 1 mg/kg, and saline alone.

FIG. 2 depicts individual categories of mecamylamine-precipitated nicotine abstinence signs over 30 minutes in subjects pretreated subcutaneously with 1 mg/kg dextromethorphan, 1 mg/kg cotinine, a combination of dextromethorphan and cotinine each as 1 mg/kg, and saline alone.

### DETAILED DESCRIPTION OF THE INVENTION

All publications, including but not limited to patents and patent applications, cited in this specification are incorporated herein by reference as though fully set forth.

Unless otherwise specified, all parts and percentages set forth herein are weight percentages based on the weight of the relevant composition. Unless otherwise stated, as used herein, the modifier “a” includes one or more of the components modified. The present invention may comprise, consist essentially of, or consist of the components set forth below, unless otherwise stated.

As used herein, the phrase “tobacco usage” means the usage of tobacco in any form by an individual, including smoking, such as cigars, cigarettes, and pipe tobacco, and using smokeless tobacco, such as snuff tobacco, spit tobacco and chewing tobacco. As used herein, the phrase “nicotine withdrawal symptoms” includes, but are not limited to, nicotine cravings, difficulty in sleeping, irritability, anxiety, restlessness, difficulty with concentration, increased appetite, weight gain and depressed mood.

An α₃β₄ nicotinic receptor antagonist as used herein means a compound that directly or indirectly blocks or otherwise reduces the activity of an α₃β₄ nicotinic receptor. Examples of α₃β₄ nicotinic receptor antagonists that are suitable for use in the present invention include, but are not limited to, mecamylamine, 18-methoxycoronaridine, bupropion, dextromethorphan, dextrorphan, and pharmaceutically acceptable salts thereof.

In one embodiment the α₃β₄ nicotinic receptor antagonist is selected from dextromethorphan, dextrorphan, and pharmaceutically acceptable salts thereof. In yet another embodiment, the α₃β₄ nicotinic receptor antagonist is selected from dextromethorphan and pharmaceutically acceptable salts thereof.

As used hereinafter, the term “dextromethorphan” refers to dextromethorphan or any of its pharmaceutically acceptable salts. As used hereinafter, the term “dextrorphan” refers to dextrorphan or any of its pharmaceutically acceptable salts.

Dextromethorphan and its active metabolite, dextrorphan are known antagonists of the N-methyl D-apartate (NMDA) receptor. In addition, dextromethorphan and dextrorphan have been shown to block the neural nicotinic receptors in the central and autonomic nervous system, such as the α₃β₄ nicotinic receptor antagonists. It has been speculated that both mechanisms may contribute to the usefulness of dextromethorphan and dextrorphan for reducing nicotine dependence. Where dextromethorphan or dextorphan is included in the compositions of the present invention, the dose range of dextromethorphan or dextorphan is from about 0.01 mg/kg to about 10 mg/kg of an individual's body weight per dose. In one embodiment the dose range of dextromethorphan or dextorphan is from about 0.01 mg/kg to about 1.6 mg/kg of an individual's body weight per dose. In yet another embodiment the dose range of dextromethorphan or dextorphan is from about 0.02 mg/kg to about 1.0 mg/kg of an individual's body weight per dose.

Metabolites of nicotine are generally derivatives of nicotine that are produced by the human body as a result of consumption, e.g., smoking, chewing, inhalation, or exposure, to a nicotine-containing material or as a result of environmental exposure to nicotine.

The term “nicotine metabolite”, as used herein, is intended to refer to any pharmacologically acceptable metabolite of nicotine which exhibits pharmacotherapeutic properties similar to nicotine. Such metabolites are known in the art, and include cotinine, norcotinine, nornicotine, nicotine 1′-N-oxide, cotinine N-oxide, cotinine glucuronide, nicotine glucuronide, trans-3′-hydroxycotinine and 5 hydroxycotinine or pharmaceutically acceptable salts thereof. In one embodiment, the metabolite of nicotine is cotinine or a pharmaceutically acceptable salt thereof.

As used hereinafter the term “cotinine” refers to cotinine or any of its pharmaceutically acceptable salts.

Cotinine has been shown to be a major metabolite of nicotine and a study by Benowitz (Clin. Pharmacol. Ther. (1983) 34(5), 604-611) estimated that 86% of systemically absorbed nicotine is metabolized to cotinine in humans. Cotinine has also been shown (Dwoskin et al, The Journal of Pharmacology and Experimental Therapeutics (1999), 288(2), 905-911) to be the most abundant metabolite in rat brain after peripheral nicotine administration. Studies also suggest that cotinine has psychological activity that can antagonize the effects of nicotine in vivo in humans. (Hatsukami et al. Psychopharmacology (1998) 135: 141-150).

In one embodiment the nicotine metabolite is cotinine. Cotinine is shown by the following structure:

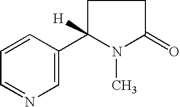

Where cotinine is present in the compositions of the present invention, the dose range of cotinine is from about 0.01 mg/kg to about 100 mg/kg of an individual's body weight per dose. In one embodiment the dose range of cotinine is from about 0.1 mg/kg to about 10 mg/kg of an individual's body weight per dose. In yet another embodiment, the dose range of cotinine is from about 0.2 mg/kg to about 3 mg/kg of an individual's body weight per dose

Where dextromethorphan or dextorphan and cotinine are both present in the compositions of the present invention, it has been found that low doses of dextromethorphan or dextorphan and cotinine may provide relief of nicotine withdrawal symptoms to an individual in need thereof. Not intending to be bound by theory, it is believed that low levels of dextromethorphan or dextorphan and cotinine which provide little or no nicotine withdrawal symptom relief when provided individually, provide surprisingly higher levels of such relief when combined in the compositions of the present invention. Thus, in yet another embodiment, the dose range of dextromethorphan or dextorphan is from about 0.02 mg/kg to about 1 mg/kg of an individual's body weight per dose and the dose range of cotinine is from about 0.2 mg/kg to about 1 mg/kg of an individual's body weight per dose.

A study was conducted to compare the effects of low doses of dextromethorphan (1 mg/kg) alone, cotinine (1 mg/kg) alone, and a dextromethorphan plus cotinine combination (1 mg/kg each) versus placebo (saline) for their effect on nicotine dependence. The subjects were 30 male Sprague-Dawley rats, weighing between 282-372 grams each. All subjects were implanted subcutaneously with an Alza 2ML1 osmotic minipump under aseptic conditions and halothane anesthesia.

All subjects were rendered nicotine dependent by seven days infusion with 9 mg/kg/day nicotine bitartrate (3.15 mg/kg expressed as the base) in saline from the subcutaneous minipump. On the 7^{th} day of infusion, each subject received two subcutaneous injections. Group 1 (n=8) received two injections of saline vehicle only. Group 2 (n=7) received a subcutaneous injection of saline and an subcutaneous injection of 1 mg/kg cotinine in saline. Group 3 (n=8) received a subcutaneous injection of saline and a subcutaneous injection of 1 mg/kg dextromethorphan. Group 4 (n=7) received a subcutaneous injection of 1 mg/kg cotinine and 1 mg/kg dextromethorphan. The cotinine and dextromethorphan doses were selected on the basis of a previous study as having only minimal effects in reducing mecamylamine-precipitated nicotine withdrawal syndrome. These low doses were intended to prevent any ceiling effect which may have masked the benefit of adding a second medication. The injection volumes in every case were 1 mg/kg.

Thirty minutes after the subcutaneous injections, each rat was challenged by 1 mg/kg subcutaneous of the nicotinic antagonist mecamylamine HCl. Each rat was then observed under blind conditions over a thirty-minute interval for precipitated nicotine abstinence signs, utilizing a standard checklist.

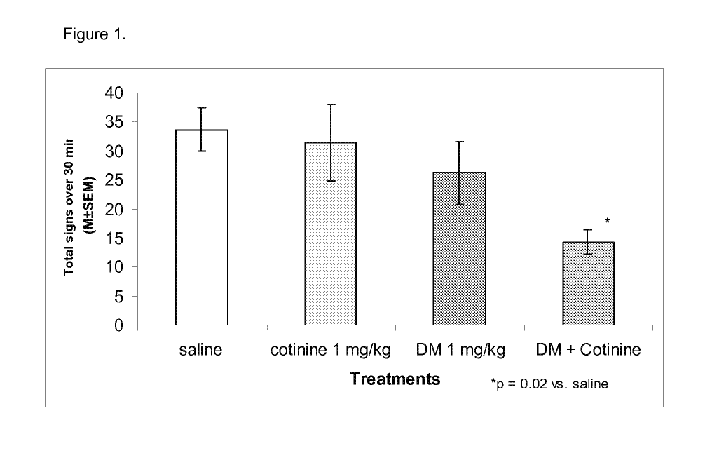

FIG. 1 depicts overall mecamylamine-precipitated nicotine abstinence signs cumulated across all categories. The combined-treatment group (cotinine plus dextromethorphan) had fewer signs than any other group.

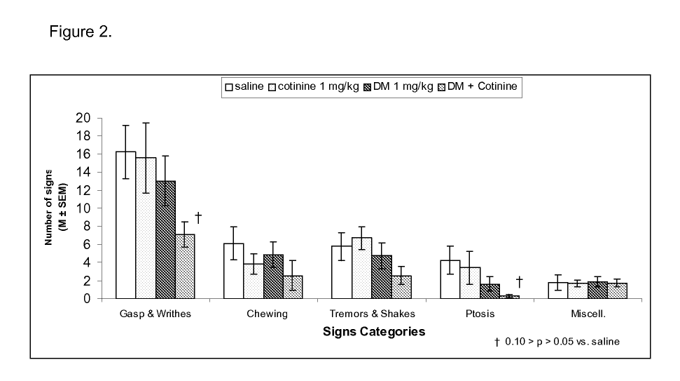

FIG. 2 depicts occurrences of individual categories of nicotine abstinence signs. In every case, the combined-treatment group had the fewest signs, except for Miscellaneous Less-Frequent Signs, where it was tied for lowest with the group receiving cotinine alone.

At the low doses tested, only the combination treatment significantly reversed nicotine dependence, as indicated by attenuation of the abstinence subsequently precipitated by 1 mg/kg mecamylamine HCl. This dose has previously been shown to precipitate a vigorous abstinence syndrome in nicotine-dependent, but not in non-dependent subject rats.

All routes of administration of the compositions of the present invention are contemplated, i.e., transdermal, oral, nasal, rectal, intravenous, intramuscular, or subcutaneous. Where the compositions are to be applied transdermally, they can be applied via a transdermal patch. For example, a transdermal patch can be configured to release an equivalent effective dose of dextromethorphan and cotinine to those described, over a period of time. Such sustained release transdermal patches can be formulated using techniques known in the art.

In one embodiment, compositions of the present invention are orally administered with absorption occurring either within the alimentary canal or via the oral or buccal mucosa of the oral cavity. Suitable oral dosage forms for compositions of the present invention include, but are not limited to; tablets, such as compressed tablets which may be coated or uncoated; caplets; hard gelatin capsules; dispersible powders; lozenges, such as hard boiled or compressed lozenges; orally dissolving strips; chewable gums; suspensions; syrups; and elixirs.

In one embodiment the composition is provided in a tablet or hard gelatin capsule dosage form. In such dosage forms, the dextromethorphan and cotinine may be mixed with an inert solid diluent, filler or bulking agent, such as, but not limited to lactose, sucralose, sucrose, glucose, fructose, trehalose, silica, dextrates, xylitol, sorbitol, mannitol, cellulose derivatives, calcium carbonate, sodium carbonate, calcium phosphate, kaolin, talc or mixtures thereof. Diluents, fillers and or bulking agents may comprise from about 25% to about 95% by weight of the total composition. In another embodiment diluents, fillers and/or bulking agents may comprise from about 50% to about 90% by weight of the total dosage form.

In addition, lubricants/glidants may be incorporated into such tablet or capsule dosage forms. Lubricants and glidants suitable for use include, but are not limited to, talc, corn starch, stearic acid, calcium stearate, polyethylene glycol, colloidal silicon dioxide, sodium stearyl fumarate, magnesium stearate vegetable and mineral oils and mixtures thereof. In one embodiment the lubricant is magnesium stearate. Where a lubricant is incorporated into these dosage forms, the lubricant may be present in an amount up to about 10% by weight of the total composition. In one embodiment the lubricant may be present in an amount up to about 5% by weight of the total dosage form.

Binding agents may also optionally be added to the dosage forms comprising the compositions of the present invention. Suitable binding agents include, but are not limited to, starch, gelatin, acacia, povidone or carbopol, or mixtures thereof. Where binding agents are incorporated into the dosage forms, they are generally present in an amount up to about 25% of the weight of the total dosage form. In one embodiment, binding agents are present up to about 10% by weight of the total dosage form.

Disintegrants may also optionally be added to the dosage forms comprising the compositions of the present invention. Suitable disintegrants include, but are not limited to, starch, alginic acid, sodium starch glycolate, or mixtures thereof. Where disintegrants are incorporated into the dosage form, they are generally present in an amount up to about 25% of the weight of the total dosage form. In one embodiment, disintegrants are present up to about 10% by weight of the total dosage form.

Additional components may be added to the compositions of the present invention including, but not limited to: flavorants, such as peppermint, spearmint, menthol, citrus, fruit flavors, vanilla, cinnamon, chocolate, coffee or tobacco flavors; colorants, such as pigments, natural food colors and dyes; sweeteners, such as the high intensity sweeteners acesulfame-K and aspartame; antioxidants/preservatives, such as sodium benzoate, butyl-hydroxy toluene and tocopherol and its salts; vitamins, such as Vitamin C or E; taste masking agents; plasticizers; and emulsifiers/surfactants.

Compositions of the present invention are useful as a tobacco replacement, and as a means to reduce or stop tobacco use. The compositions may be used as a total or partial replacement of tobacco, and may be used concurrently with tobacco as part of a planned tobacco reduction program, e.g., while reducing tobacco usage prior to outright quitting tobacco usage.

The present invention also relates to methods of reducing tobacco usage, comprising administering a composition of the present invention to a person in need thereof. The present invention also relates to a method of reducing nicotine withdrawal symptoms comprising administering the compositions of the present invention to a person in need of such relief. “Need” is intended to include a person's desire to reduce tobacco usage or nicotine withdrawal symptoms, respectively. “Reducing” nicotine withdrawal symptoms or tobacco usage includes eliminating nicotine withdrawal symptoms or tobacco usage.

### EXAMPLES

Without further elaboration, it is believed that one of skill in the art can, using the preceding description, utilize the present invention to its fullest extent. The following examples, therefore, are to be construed as merely illustrative and not a limitation of the scope of the present invention.

### Examples 1 & 2

### A Compressed Tablet Formulation

All components are screened and subsequently mixed. The resultant mixture is then compressed by any methods known in the art, such as direct compression with standard equipment well known in the art, to an appropriate hardness, for orally administrated tablets:

| | Example 1 | | Example 2 | |
|---|---|---|---|---|
| | % by | Weight | % by | Weight |
| Ingredients | Weight | (mg/tablet) | Weight | (mg/tablet) |
| Cotinine | 12% | 60 | 0.12% | .6 |
| Dextromethorphan | 12% | 60 | 0.24% | 1.2 |
| Mannitol (Filler) | 64% | 320 | 87.64% | 438.2 |
| Carbopol (Binder) | 5% | 25 | 5% | 25 |
| Magnesium Stearate | 2% | 10 | 2% | 10 |
| (Lubricant) | | | | |
| Sodium Starch | 5% | 25 | 5% | 25 |
| Glycolate | | | | |
| (disintegrant) | | | | |
| Totals | 100% | 500 | 100% | 500 |

What is claimed is:

1. A composition for treating nicotine dependence comprising:
a) at least one α₃β₄ nicotinic receptor antagonist; and
b) at least one metabolite of nicotine.

2. The composition of claim 1, wherein the α₃β₄ nicotinic receptor antagonist is selected from the group consisting of mecamylamine, 18-methoxycoronardine, burpoprion, dextromethorphan, dextrorphan, and pharmaceutically acceptable salts thereof.

3. The composition of claim 2 wherein the α₃β₄ nicotinic receptor antagonist is selected from the group consisting of dextromethorphan, dextrorphan, and pharmaceutically acceptable salts thereof.

4. The composition of claim 3 wherein metabolite of nicotine is selected from the group consisting of cotinine, norcotinine, nornicotine, nicotine N-oxide, cotinine N-oxide, 3-hydroxycotinine and 5-hydroxycotinine and pharmaceutically acceptable salts thereof.

5. The composition of claim 3 wherein the α₃β₄ nicotinic receptor antagonist is dextromethorphan.

6. The compositions of claim 5 wherein the at least one metabolite of nicotine is cotinine.

7. The composition of claim 6 wherein the dose range of cotinine is from about 0.01 mg/kg to about 100 mg/kg of an individual's body weight per dose.

8. The composition of claim 7 wherein the dose range of cotinine is from about 0.1 mg/kg to about 10 mg/kg of an individual's body weight per dose.

9. The composition of claim 8 wherein the dose range of cotinine is from about 0.2 mg/kg to about 3 mg/kg of an individual's body weight per dose.

10. The composition of claim 9 wherein the dose range of dextromethorphan is from about 0.01 mg/kg to about 10 mg/kg of an individual's body weight per dose.

11. The composition of claim 10 wherein the dose range of dextromethorphan is from about 0.01 mg/kg to about 1.6 mg/kg of an individual's body weight per dose.

12. The composition of claim 11 wherein the dose range of dextromethorphan is from about 0.02 mg/kg to about 1 mg/kg of an individual's body weight per dose.

13. The composition of claim 1 wherein the composition is adapted for oral administration.

14. The composition of claim 13 wherein the composition is an oral dosage form selected from the group consisting of tablets, caplets, hard gelatin capsules, lozenges, orally dissolving film and chewable gums.

15. A method of reducing nicotine withdrawal symptoms comprising administrating a composition of claim 1 to an individual in need thereof.

16. A method of reducing nicotine withdrawal symptoms comprising administrating a composition of claim 6 to an individual in need thereof.

17. A method of reducing nicotine withdrawal symptoms comprising administrating a composition of claim 12 to an individual in need thereof.

18. A method of reducing tobacco usage comprising administrating a composition of claim 1 to an individual in need thereof.

19. A method of reducing tobacco usage comprising administrating a composition of claim 6 to an individual in need thereof.

20. A method of reducing tobacco usage comprising administrating a composition of claim 12 to an individual in need thereof.
